(19) United States
(12) Patent Application Publication
(10) Pub. No.: US 2026/0294252 A1
(43) Pub. Date: Oct. 1, 2026

(54) TISSUE DETECTION SYSTEMS AND METHODS

(71) Applicant: Medtronic, Inc., Minneapolis, MN (US)

(72) Inventor: Zhigang Shi, Shanghai (CN)

(21) Appl. No.: 19/489,369

(22) PCT Filed: Jun. 9, 2023
(86) PCT No.: PCT/CN2023/099374

§ 371 Date: Dec. 2, 2025

(51) Int. Cl. A61B 5/00; G06V 10/14; G06V 10/70

(52) U.S. Cl. CPC A61B 5/0071; A61B 5/0077; A61B 5/7221; G06V 10/14; G06V 10/70; G06V 2201/03

(57) ABSTRACT

A tissue detection system (10) includes an emitter (105) configured to generate an electromagnetic radiation signal for application to tissue, a detector (110) configured to detect fluorescence of the tissue responsive to application of the electromagnetic radiation signal to the tissue, and a controller (140) configured to detect a tissue of interest within the tissue based upon the detected fluorescence and to determine a confidence associated with the detection of the tissue of interest. If the determined confidence meets a threshold, the controller (140) is configured to output an indication of the detected tissue of interest. If, on the other hand, the determined confidence fails to meet the threshold, a computing device (300) is configured to at least one of: further detect the tissue of interest or confirm the detection of the tissue of interest.

### BACKGROUND

### Technical Field

The present disclosure relates to tissue detection and, more particularly, to systems and methods facilitating detection of tissue of interest at a surgical site.

### Background of Related Art

Many surgical procedures are performed at surgical sites on or within the body where the detection of tissue of interest via direct visualization techniques alone (e.g., using the human eye, a lens-based endoscope, a surgical video camera, etc.) is difficult due to obstructions, darkness, minimal or no contrast between different tissues, minimal or no visible distinction between different tissues, etc. Such surgical procedures may thus benefit from the use of enhanced visualization techniques such as, for example, fluorescence.

Since some materials, including certain tissues, fluoresce when stimulated with electromagnetic radiation (e.g., light at non-visible wavelengths), fluorescence can be used to highlight tissue of interest, thus facilitating detection of tissue of interest that may otherwise be difficult or impossible to detect solely by direct visualization techniques. The particular wavelength or wavelengths of electromagnetic radiation emitted and detected may depend upon the tissue or tissues of interest to be highlighted.

### SUMMARY

To the extent consistent, any or all of the aspects detailed herein may be used in conjunction with any or all of the other aspects detailed herein.

Provided in accordance with aspects of the present disclosure is a tissue detection system including an emitter configured to generate an electromagnetic radiation signal for application to tissue, a detector configured to detect fluorescence of the tissue responsive to application of the electromagnetic radiation signal to the tissue, and a controller configured to detect a tissue of interest within the tissue based upon the detected fluorescence and to determine a confidence associated with the detection of the tissue of interest. If the determined confidence meets a threshold, the controller is configured to output an indication of the detected tissue of interest. If, on the other hand, the determined confidence fails to meet the threshold, a computing device is configured to at least one of: further detect the tissue of interest or confirm the detection of the tissue of interest.

In an aspect of the present disclosure, the computing device is remote from the controller and configured to communicate with the controller over a network.

In another aspect of the present disclosure, the computing device is configured to perform the at least one of the further detecting of the tissue of interest or the confirming of the detection of the tissue of interest via image processing.

In still another aspect of the present disclosure, the system further includes at least one camera configured to capture an image of the detected tissue of interest. The computing device is configured to perform the at least one of the further detecting of the tissue of interest or the confirming of the detection of the tissue of interest via image processing of the image captured by the at least one camera.

In yet another aspect of the present disclosure, the at least one camera includes at least one of a visible camera or an infrared (IR) camera.

In still yet another aspect of the present disclosure, the computing device is configured to perform the at least one of the further detecting of the tissue of interest or the confirming of the detection of the tissue of interest using machine learning and, in aspects, via machine learning image processing.

In another aspect of the present disclosure, the computing device is a cloud server.

In aspects of the present disclosure, the confidence is numerical or categorical.

In yet another aspect of the present disclosure, the system further includes a probe configured to deliver the electromagnetic radiation signal from the emitter to the tissue. In such aspects, the probe may be coupled between the tissue and the detector to enable the detector to detect the fluorescence of the tissue responsive to the application of the electromagnetic radiation signal to the tissue through the probe. The detector may alternatively be independent of the probe.

In still another aspect of the present disclosure, the probe includes a camera configured to produce image data for use by the computing device in the at least one of the further detecting of the tissue of interest or the confirmation of the detection of the tissue of interest.

In still yet another aspect of the present disclosure, the system further includes a camera independent of the probe and configured to produce image data for use by the computing device in the at least one of the further detecting of the tissue of interest or the confirmation of the detection of the tissue of interest.

A method of tissue detection provided in accordance with aspects of the present disclosure includes applying a signal to tissue, detecting a response of the tissue from the application of the signal to the tissue, detecting a tissue of interest within the tissue based upon the detected response, determining a confidence associated with the detection of the tissue of interest, outputting an indication of the detected tissue of interest if the determined confidence meets a threshold, and further detecting the tissue of interest or confirming the detection of the tissue of interest if the determined confidence fails to meet the threshold.

In an aspect of the present disclosure, the detecting is performed locally and the further detecting or the confirming is performed remotely.

In another aspect of the present disclosure, the detecting is performed with a traditional algorithm and the further detecting or the confirming is performed with a machine learning algorithm.

In still another aspect of the present disclosure, the detecting is performed based on evaluating the response for fluorescence and the further detecting or the confirming is performed based on image processing of an image of the tissue.

In yet another aspect of the present disclosure, the signal is an electromagnetic radiation signal, the response is the fluorescence of the tissue, and wherein, if the determined confidence fails to meet the threshold, the method further includes capturing the image of the tissue for the image processing of the image of the tissue.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a tissue detection system in accordance with the present disclosure illustrated in use in relation to a patient and an operator, where an internal area of the patient is enlarged for reference;

FIG. 2 is a schematic illustration of a probe, emitter, detector, controller, and user interface of the tissue detection system of FIG. 1;

FIG. 3 is a block diagram of the probe, the controller, the user interface, a camera system, a display, and a computing system of the tissue detection system of FIG. 1 illustrated in use in relation to a patient;

FIG. 4 is a block diagram of implementation of a machine learning algorithm by the computing system of the tissue detection system of FIG. 1;

FIG. 5 is a schematic illustration of an exemplary convolutional neural network (CNN) configured for implementation by the computing system of the tissue detection system of FIG. 1; and

FIG. 6 is a flow diagram of a method of detecting tissue in accordance with the present disclosure.

### DETAILED DESCRIPTION

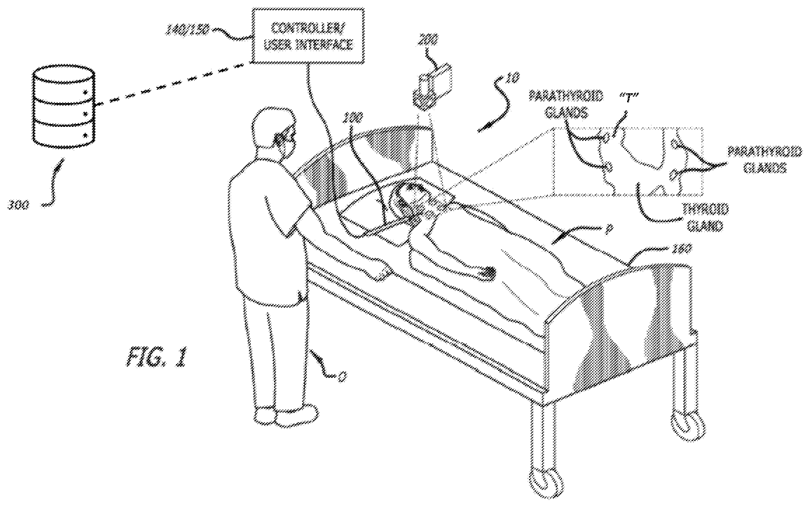

In the following description, well-known functions or constructions are not described in detail to avoid obscuring the present disclosure in unnecessary detail. Those skilled in the art will understand that the systems and methods of the present disclosure may be performed by one or more operators “O” (FIG. 1), which may be one or more human clinicians and/or one or more surgical robots.

The tissue detection systems and methods of the present disclosure may be utilized in surgical procedures to detect tissue (via affirmative or negative identification relative to surrounding tissue) and, if applicable, facilitate performing a surgical procedure on and/or around the detected tissue. For example, the tissue detection systems and methods of the present disclosure may be utilized to detect parathyroid tissue (e.g., within thyroid tissue), thyroid tissue, and/or other tissues in the neck region to facilitate removal or treatment of such tissue or surrounding tissue during surgery, or to avoid such tissue when removing or treating other tissue during surgery. However, although the aspects and features of the present disclosure are described hereinbelow with respect to detecting tissue in the neck region, e.g., parathyroid tissue and/or thyroid tissue, the aspects and features of the present disclosure are equally adaptable for use in the detection of different tissue and/or tissue at different anatomical locations. That is, although different instrumentation may be required to access different tissue and/or different anatomical locations, and although different settings, e.g., different electromagnetic radiation wavelengths, may be required to identify different tissue, the aspects and features of the present disclosure remain generally consistent regardless of the particular instrumentation and/or settings utilized.

Referring to FIG. 1, a tissue detection system 10 provided in accordance with aspects of the present disclosure generally includes a probe 100, a controller 140, and a user interface 150. Tissue detection system 10 further includes a camera system 200 that can include an Infrared (IR) camera to capture fluorescence (as still IR images and/or an IR video feed) from fluorescing tissue of interest and/or a visible camera (to capture still visible images and/or a visible video feed of tissue of interest). Further, tissue detection system 10 includes or is connected to a computing system 300 that communicates with controller 140, e.g., through a wired or wireless connection. Computing system 300 can include one or more computing devices (e.g., desktop computers, laptop computers, tablets, smartphones, servers (such as local servers, remote servers, and/or cloud servers), combinations thereof, and/or any other suitable computing devices) connected to one another and controller over a communication link such as a network accessible via the internet or an intranet.

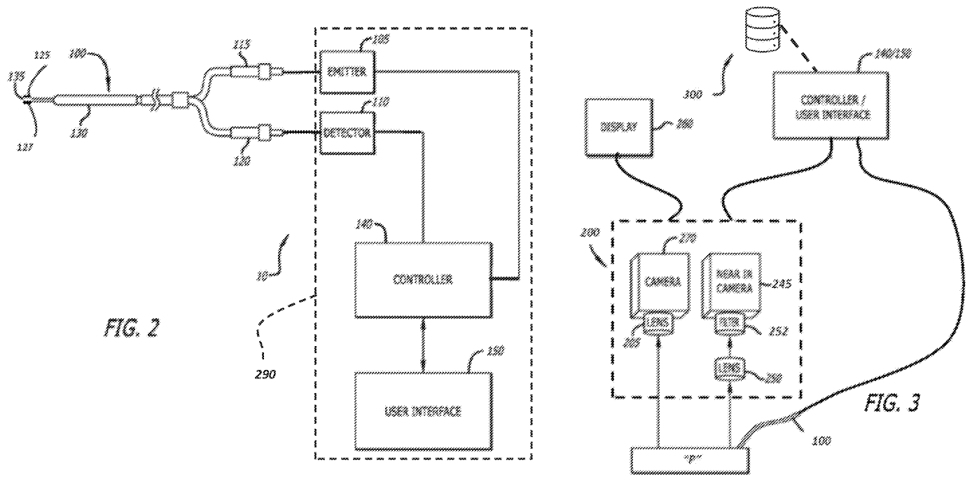

Referring still to FIG. 1, probe 100 can be positioned by an operator “O” relative to a patient “P” received on a surgical table 160. Although the operator “O” is shown as a human clinician, it is also contemplated that the operator “O” is a surgical robot. Probe 100 is configured to be maneuvered into position, e.g., by operator “O,” into contact or close proximity (e.g., within about 5 cm) and directed at tissue of interest such as parathyroid tissue “T” of patient “P.” Probe 100 operably connects to one or more emitters 105 (FIG. 2) configured to direct electromagnetic radiation from probe 100 to the tissue of interest to stimulate the tissue of interest such that any fluorescence produced by the stimulated tissue can be detected by one or more detectors 110 (FIG. 2), which may be operably coupled to probe 100, incorporated into or operably coupled to camera system 200, and/or separately provided.

Camera system 200, and as mentioned above, is configured to detect fluorescence and/or to obtain visible images. To this end, camera system 200 may include, for example, an IR camera 245 (FIG. 3), such as, for example, a near IR camera, and/or a visible camera 270 (FIG. 3). Camera system 200 may be positioned spaced-apart from the surgical site as compared to probe 100, such that cameral system 200 may provide fluorescence detection and/or visible imaging over a relatively large field of view. In such aspects, combining use of camera system 200 with probe 100 enables fluorescence detection by camera system 200 to identify potentially fluorescing tissue over the relatively large field of view, and enables probe 100 to be used for fluorescence detection locally, within the relatively focused field of view thereof, at the location of each of the potentially fluorescing tissues, e.g., by positioning probe 100 in contact with or in close proximity (e.g., within about 5 cm) to the surface of each of the potentially fluorescing tissues, to enable confirmation as to whether the potentially fluorescing tissue identified by camera system 200 is indeed fluorescing.

Continuing with reference to FIG. 1, controller 140 and user interface 150 may be incorporated into a single integrated unit, may be physically connected or connectable with one another, or may be separate from one another. Controller 140 and/or user interface 150 can include a display or be connectable to a display, e.g., display 260 (FIG. 3), for displaying information obtained through use of tissue detection system 10 such as, for example, IR images of fluorescing tissue and/or visible images of tissue.

Controller 140 includes a processor to process data, a memory in communication with the processor to store data, and an input/output unit (I/O) to interface with other modules, units, and/or devices. The processor can include a central processing unit (CPU), a microcontroller unit (MCU), or any other suitable processor or processors. The memory can include and store processor-executable code, which when executed by the processor, causes controller 140 to perform various operations, e.g., such as receiving information, commands, and/or data, processing information and data, and transmitting or providing information/data to another device. To support various functions of controller 140, the memory can store information and data, such as instructions, software, values, images, and other data processed or referenced by the processor. For example, various types of Random-Access Memory (RAM) devices, Read Only Memory (ROM) devices, Flash Memory devices, and other suitable storage media can be used to implement storage functions of the memory. The I/O of controller 140 enables controller 140 to interface with other devices or components of devices utilizing various types of wired or wireless interfaces (e.g., a wireless transmitter/receiver (Tx/Rx)) compatible with typical data communication standards to enable communication between controller 140 and other devices, e.g., user interface 150, display 260 (FIG. 3), computing system 300, etc. Examples of typical data communication standards include, but are not limited to, Bluetooth, Bluetooth low energy, Zigbee, IEEE 802.11, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/5G/LTE cellular communication methods, NFC (Near Field Communication), and parallel interfaces. The I/O of controller 140 can also interface with other external interfaces, sources of data storage, and/or visual or audio display devices, etc. to retrieve and transfer data and information that can be processed by the processor, stored in the memory, and/or output to an external device. In aspects, for example the output may be provided to various types of screen displays, speakers, or printing interfaces, e.g., including but not limited to, LED, liquid crystal display (LCD) monitor or screen, cathode ray tube (CRT), audio signal transducer apparatuses, and/or toner, liquid inkjet, solid ink, dye sublimation, inkless (e.g., such as thermal or UV) printing apparatuses, etc.

User interface 150 may include, for example, any of the output devices noted above, a display, Graphical User Interface (GUI), a touch-screen GUI, a keyboard, a mouse, physical and/or digital buttons, a speaker, one or more LED lights, a foot switch, a hand switch, and/or any other suitable interface devices to enable the input of information, e.g., to control the operation of system 10, and/or to output information, e.g., regarding the status and/or result of the operation of system 10. For example, user interface 150 may: include a suitable input to enable the activation of probe 100, e.g., to emit electromagnetic radiation; include a suitable input (the same or different from the above input) to enable the activation of fluorescence detection, e.g., via probe 100 and/or camera system 200; and/or may provide a perceptible output, e.g., audio, visual, tactile, indicating that a suitable fluorescence signal has been detected.

With additional reference to FIG. 2, probe 100, in aspects, may include one or more probe bodies 130 each including one or more emitter optical fibers 115 coupled to one or more emitters 105 and/or one or more detector optical fibers 120 coupled to one or more detectors 110. Additional or alternative detection may be provided by camera system 200 (FIG. 1), as detailed below. Although plural of the components are contemplated, probe body 130, emitter optical fiber 115, emitter 105, detector optical fiber 120, and detector 110 are described herein below in the singular to facilitate understanding. In aspects, probe 100, or at least probe body 130 thereof, may be integrated (permanently or removably) within a surgical endoscope (not shown) or other surgical device. Emitter 105 and detector 110 may be integrated into a single unit, e.g., a console 290 including a housing, or may be separate from one another. Whether integrated or separate, emitter 105 and/or detector 110 may be integrated with or separate from controller 140 and/or user interface 150. For example, console 290 may include emitter 105, detector 110, controller 140 and/or user interface 150 incorporated therein or thereon. In further aspects, display 260 (FIG. 3) is also incorporated into or onto console 290.

Emitter 105 is configured to emit electromagnetic radiation at a particular wavelength or within a particular wavelength range, e.g., via tuning and/or equipment selection, through emitter optical fiber 115 and out a distal end portion 135 of probe body 130 (either axially therefrom, transversely therefrom, or in any other suitable direction or directions including adjustable directions) in order to stimulate fluorescence of a particular tissue or tissues of interest. With respect to identification of parathyroid tissue, for example, emitter 105 (with or without the use of one or more optical elements 125 disposed at the output end of emitter optical fiber 115 at distal end portion 135 of probe body 130) may be configured to emit electromagnetic radiation in the form of laser energy at a wavelength of about 785 nm to facilitate autofluorescence of parathyroid tissue. Emitter 105, at least for use in identifying parathyroid tissue, may be a narrow band source such as a laser (e.g., a solid state laser, a laser diode, etc.) or other suitable source whose electromagnetic radiation output wavelength is at or near a narrow band around about 785 nm. Tuning, equipment selection, and/or filtering (using one or more optical elements 125, e.g., a band-pass (BP) filter, disposed at the output end of emitter optical fiber 115 at distal end portion 135 of probe body 130) may be utilized to facilitate achieving this narrow band. Of course, for identification of different tissues, different narrow (or broader) wavelength bands may be utilized and, as a result, different tuning, equipment selection, and/or optical elements 125 may be provided. Optical elements 125 may alternatively or additionally be disposed at different locations other than at distal end portion 135 of probe body 130 and may include, for example, lenses, filters, mirrors, beamsplitters, etc. Controller 140 can be used to control transmission, e.g., activate/deactivate, control the wavelength, intensity, etc., of the electromagnetic radiation from emitter 105 to tissue of interest (via emitter optical fiber 115). User interface 150 can be used to interact with and control operation of the controller 140 (e.g., to set parameters and/or activate/deactivate), which in turn controls emitter 105.

Detector 110 is configured to detect fluorescence of the tissue of interest (as a result of the electromagnetic radiation emitted to stimulate the tissue of interest) collected at distal end portion 135 of probe body 130 and transmitted through detector optical fiber 120 to detector 110. Detector 110 is further configured to process the received fluorescence signal. Controller 140 may be utilized to control and/or facilitate processing of the detected fluorescence signal at detector 110. With respect to detection of parathyroid tissue, for example, detector 110 may be configured to process the fluorescence signal, which for parathyroid tissue undergoing autofluorescence is at wavelengths ranging from about 808 nm to about 1000 nm. Detector 110 may be an avalanche photodiode or other near IR detector, a 2D array of IR detectors, or other suitable detector, and may be used in concert with one or more optical elements 127, e.g., a longpass (highpass) optical filter, such that radiation wavelengths above the source wavelength (for instance, above about 800 nm, e.g., ranging from about 808 to about 1000 nm) can be detected with minimal interference from other non-relevant wavelengths of electromagnetic radiation, e.g., such as from ambient light. Reducing the effects of ambient light may also be accomplished by positioning probe body 135 in contact with or close proximity (e.g., within about 5 cm) to the tissue of interest during emission/detection; by modulating the emitter radiation; and/or by collecting the fluorescence signal using a phase lock technique, e.g., lock-in detection or FFT (fast Fourier transform) techniques. With respect to the one or more optical elements 127 (such as a Long-Pass (LP) filter, for example), such optical elements 127, in aspects, are provided at the input end of detector optical fiber 120 at distal end portion 135 of probe body 130. Alternatively or additionally, the one or more optical elements 127 may be disposed at the input to detector 110, e.g., at the output end of detection optical fiber 120 or between the output end of detection optical fiber 120 and the input to detector 110.

A detected fluorescence signal, e.g., obtained and processed by detector 110, for a tissue of interest may be evaluated by controller 140 by comparing the detected fluorescence signal with a baseline fluorescence signal to determine if the detected fluorescence signal is indicative of the presence of a particular tissue, or may be processed in any other suitable manner. Details with respect to systems and methods using autofluorescence for discriminating parathyroid tissue from thyroid tissue or other tissues in a neck region are described in U.S. Pat. No. 9,687,190 titled “Intra-Operative Use of Fluorescence Spectroscopy and Applications of Same,” the entire contents of which are hereby incorporated by reference herein. As disclosed therein, when the thyroid tissue and the parathyroid tissue are exposed to radiation in a narrow wavelength range of about 785 nm, which is just outside the visible light range, both the thyroid tissue and the parathyroid tissue produce autofluorescence in a wavelength range above about 800 nm, sometimes centered at about 822 nm (the wavelength range above about 800 nm is also not visible). However, the intensity of the autofluorescence of the parathyroid tissue is significantly higher than that of the thyroid tissue, enabling distinction between these two tissues and, thus, detection of the parathyroid tissue within the thyroid tissue. More specifically, the detection of the parathyroid tissue within the thyroid tissue may be determined by controller 140, for example, based on a ratio of the intensity of the detected fluorescence signal to the intensity of the baseline fluorescence signal. With respect to areas where the intensity exceeds a threshold or other criteria, those areas may be identified as parathyroid tissue. These systems and methods may also be applied for use in detecting other tissues (with appropriate adjustment of the wavelengths and baseline signals). Other suitable systems methods for detecting tissue, e.g., parathyroid tissue, are also contemplated.

In addition to detecting tissue (for example, parathyroid tissue within thyroid tissue) based on a comparison of the detected fluorescence signal with the baseline fluorescence signal (e.g., using a ratio of the intensities thereof), controller 140 is further configured to determine a confidence associated with the tissue detection. This confidence may be, in aspects, a numerical value indicating a confidence in the tissue detection such as, for example: a confidence number on a numerical scale (e.g., 1-10); a confidence percentage on a percentile scale (e.g., 0-100%); or any other suitable numerical value. Alternatively or additionally, the confidence may be a categorical determination indicating a confidence in the tissue detection such as, for example: a binary determination of sufficient or insufficient (YES/NO) confidence in the tissue detection; a determination of a confidence level, e.g., high confidence, moderate confidence, or low confidence in the tissue detection, or any other suitable categorical determination of confidence associated with the tissue detection.

The confidence in the tissue detection, whether numerical, categorical, or provided in any other suitable manner, may be determined, in aspects, based on the ratios of the intensities of the detected fluorescence signals to the intensities of the baseline fluorescence signals. For example, where the ratios equal or minimally exceed a threshold for detecting the tissue, a lower or relatively low confidence may be assigned. On the other hand, where the ratios clearly exceed the threshold for detecting the tissue, a higher or relatively higher confidence may be assigned. The confidence in the tissue detection may additionally or alternatively be assigned by statistical confidence interval determination and/or in other suitable manner.

The confidence in the tissue detection, as detailed below, is utilized to determine whether further action is required to enable accurate detection of the tissue of interest. For example, where the confidence meets a confidence threshold (whether a numerical threshold (e.g., at least 80% confidence) or a categorial threshold (e.g., high confidence)), tissue detection is determined to have been accomplished with sufficient confidence such that no further detection or confirmation is required. On the other hand, where the confidence is below the confidence threshold, tissue detection is determined to have been accomplished with insufficient confidence such that further detection or confirmation is required. Evaluating the confidence in the tissue detection before determining whether to proceed with further detection or confirmation saves time and resources by avoiding the need for further detection or confirmation in instances where the tissue detection is made with sufficient confidence while still enabling the use of further detection or confirmation to provide more accurate tissue detection in instances where the tissue detection is made with insufficient confidence. In aspects, as detailed below, while the initial tissue detection is performed locally e.g., via controller 140, in first manner, e.g., using fluorescence, the further detection or confirmation may be performed remotely, e.g., via computing device 300 (FIGS. 1 and 3), and/or in a second, different manner, e.g., using image processing. Additionally or alternatively, while the initial tissue detection is performed utilizing a traditional algorithm(s), the further detection or confirmation may be performed utilizing a machine learning algorithm(s).

Turning to FIG. 3, camera system 200 is shown in use on a patient “P” together with probe 100, controller 140, user interface 150, display 260, and computing system 300. Camera system 200 includes, as noted above, an IR camera (e.g., a near IR camera 245) and/or a visible camera 270.

One or more optical elements, e.g., one or more lens 250 and/or one or more filters 252, may be positioned to modify the fluorescence signal to be received by near IR camera 245. Near IR camera 245, with or without use of optical elements 250, 252, captures any fluorescence radiation (e.g., resulting from the stimulation of the tissue of interest by the emitted electromagnetic radiation provided by emitter 105 via probe 100) within its field of view, which includes the tissue of interest. The fluorescence signal obtained by near IR camera 245 may, in aspects, be processed, e.g., passed through a demodulator. With or without processing the fluorescence signal obtained by near IR camera 245 is then transmitted to display 260 and/or controller 140. Controller 140 (or a separate controller associated within camera system 200) may detect tissue based upon the fluorescence signal obtained by near IR camera 245, e.g., similarly as detailed above or in any other suitable manner. Controller 140 (or the separate controller associated within camera system 200) may further determine a confidence in the detection of tissue, e.g., similarly as detailed above or in any other suitable manner. In aspects where tissue is detected using both detector 110 (FIG. 2) and IR camera 245, the confidence determination may be based on a comparison of the tissue detection by detector 110 (FIG. 2) and that of IR camera 245, e.g., wherein similar tissue detection results correspond to higher confidence and wherein different tissue detection results correspond to lower confidence.

Visible video camera 270 may be provided as part of camera system 200 in addition to or as an alternative to near IR camera 245. Visible video camera 270 may include a lens 205 to modify the image signal to be received by visible video camera 270. Cameras 245, 270, where both provided, may be integrated into a single unit, connected to or connectable with one another, or separate from one another. Further, visible video camera 270 may be configured to image overlapping and, in aspects, coextensive fields of view with near IR camera 245 (if both are provided).

In aspects, rather than a separate camera system 200, IR camera 245 and/or visible camera 270 may be integrated into probe 100, e.g., probe 100 itself or as part of an endoscope or other surgical device releasably or permanently securing probe 100 and one or both of cameras 245, 270 relative to one another.

Display 260, which may be configured as any of the above-noted output devices, above-noted user interface devices, or any other suitable device for displaying information, can be separate from or integrated with controller 140 and/or user interface 150, e.g., incorporated into or connected with console 290 (FIG. 2). Camera system 200 may output, to display 260, still and/or video images for display, e.g., substantially real time display, of any visible tissue and/or fluorescing tissue. With respect to fluorescing tissue, the display may include an indication of the wavelength and/or intensity of such fluorescence, e.g., via highlighting, color change, or in any other suitable manner. Further, processing of the near IR fluorescence signals from near IR camera 245 together with the video signals from visible video camera 270 may enable pixel matching such that fluorescing tissue (as detected by near IR camera 245) can be highlighted on the visible images produced by visible video camera 270. The highlighted video image may ultimately be displayed on display 260 and/or another suitable display such that the highlighted tissue on the substantially real-time visible video image can be readily identified. Highlighted or otherwise annotated still images are also contemplated. Other suitable techniques for highlighting fluorescing tissue on substantially real-time video images and/or still images, overlaying fluorescing tissue images onto substantially real-time video images and/or still images, or otherwise indicating fluorescing tissue on substantially real-time video images and/or still images may also be provided.

In aspects, display of the fluorescing tissue, highlighting fluorescing tissue, and/or otherwise outputting an indication of the detected tissue may be provided automatically. Alternatively, indication(s) of the detected tissue may only be provided where it determined that the tissue detection was made with sufficient confidence. In such aspects, where it is determined that the tissue detection was not made with sufficient confidence, the indication(s) of the detected tissue may be withheld unless or until further detection or confirmation is provided to ensure appropriate tissue detection.

Referring generally to FIGS. 1-3, as noted above, tissue detection system 10 not only detects tissue using fluorescence, but also evaluates the confidence in the tissue detection. If it is determined, based on the confidence in the tissue detection meeting a confidence threshold, that the tissue detection was made with sufficient confidence, the detected tissue may be displayed (with suitable highlight or other visual indicators) and/or otherwise indicated to the operator “O,” e.g., without further intervention. On the other hand, if it is determined, based on the confidence in the tissue detection failing to reach the confidence threshold, that the tissue detection was made with insufficient confidence, further detection or confirmation may be performed to ensure accurate tissue detection, e.g., prior to display and/or other indication of the same to the operator “O.”

The further detection or confirmation may be performed by computing system 300 utilizing, for example, one or more visible images of the tissue of interest and/or one or more IR images of the tissue of interest. More specifically, where controller 140 determines that the tissue detection was made with insufficient confidence, controller 140 may direct the capture and/or output of one or more images, e.g., IR images captured by camera 245 and/or visible images captured by camera 270, to computing system 300. Computing system 300 may be local, remote, in the cloud, and/or distributed amongst several local, remote, and/or cloud devices. Communication of the image data, e.g., of the images captured by camera 245 and/or camera 270, from controller 140 to computing system 300 may be made over any suitable communication link.

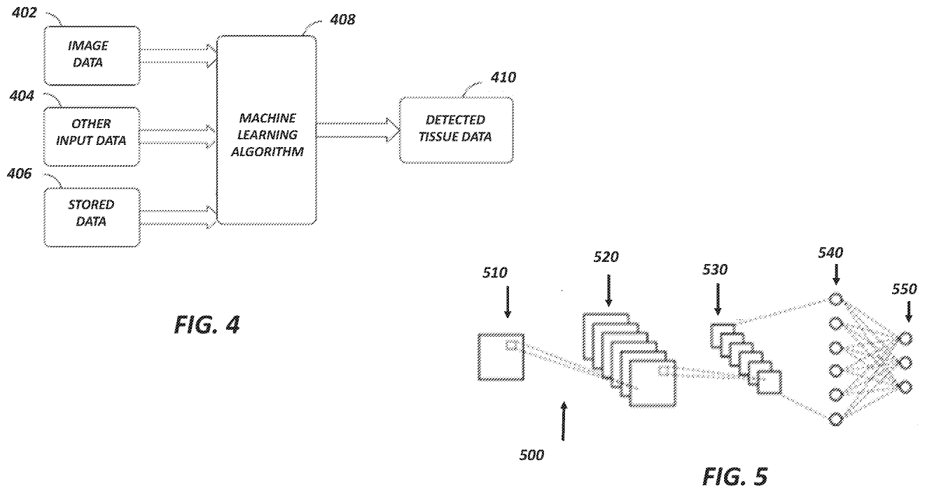

With additional reference to FIG. 4, computing system 300, in addition to receiving the image data 402 (e.g., captured by camera 245 and/or camera 270), may also obtain other input data 404 and/or access stored data 406. The other input data 404 may be obtained by computing system 300 from tissue detection system 10 or another suitable source, e.g., a patient's electronic medical record (EMR), hospital database, operator-input information, etc. The other input data 404 may include, for example, information about the patient (demographic information, biometric information, medical conditions, etc.), the procedure to be performed, the equipment, devices and/or systems utilized, the operator, etc. The other input data 404 may additionally or alternatively include the fluorescence data (e.g., the raw fluorescence data or the determined ratios of fluorescence intensity data) from tissue detection system 10 and/or the detected tissue data as determined by controller 140 of tissue detection system 10. The stored data 406 may include, for example, a catalogue of images of known tissues and/or data correlating known images with corresponding tissues. The stored data 406 may be stored on computing system 300 or separately therefrom.

Computing system 300 is configured to input the image data 402, along with the other input data 404 and/or the stored data 406, into a machine learning algorithm 408. The machine learning algorithm 408 is configured to output detected tissue data 410 identifying the tissue of interest within the input images and/or confirming whether the initial detection of the tissue of interest was accurate. Machine learning algorithm 408 may be utilized by implementing one or more of: supervised learning, semi-supervised learning, unsupervised learning, reinforcement learning, association rule learning, decision tree learning, anomaly detection, feature learning, etc., and may be modeled as one or more of a neural network, Bayesian network, support vector machine, genetic algorithm, etc. The machine learning algorithm(s) may be trained based on empirical data and/or other suitable data and may be trained prior to deployment for use during a surgical procedure or may continue to learn based on usage data after deployment and use in a surgical procedure(s). In aspects, image data associated with tissue detection that was made with sufficient confidence (or a higher threshold of confidence) may be transmitted to computing system 300 for use in the training data for training machine learning algorithm 408. That is, controller 140 (FIGS. 2-3) may direct the transmission of image data to computing system 300 in instances where the tissue detection was performed with sufficient confidence not for further detection or confirmation but for use as training data for machine learning algorithm 408.

With respect to neural networks, convolutional neural networks (CNNs) are generally accepted as very efficient and effective deep learning algorithms for image processing machine learning applications. Referring to FIG. 5, an exemplary CNN 500 is shown. CNN 500 includes an input layer 510 (configured to receive the data 402, 404, 406), one or more convolutional layers 520, one or more pooling layers 530, one or more fully connected layers 540, and an output layer 550 configured to output an identification of the tissue of interest. In aspects, a flattening layer (not explicitly shown) is disposed between the final pooling layer 530 or final convolutional layer 520 and the first fully connected layer 540. Further, a pooling layer 530 may be disposed after each convolutional layer 520 or a set of convolutional layers 520. In other configurations, pooling layer 530 is omitted.

The one or more convolutional layers 520 may implement any suitable similar or different activation functions (e.g., ReLU or Tanh). Further the one or more convolutional layers 520 may include any suitable number of kernels, e.g., 32, 64, or 128; may implement a Sobel filter or other suitable filter; may utilize any suitable filter size, e.g., 3×3, 5×5, or 7×7; and/or may utilize any suitable stride, e.g., 1 or 2. Padding, e.g., of 1 or 2, may also be utilized, in aspects.

The one or more pooling layers 530 may be similar or different and may utilize, for example, max pooling or average pooling.

The one or more fully connected layers 540 may use any suitable similar or different activation functions (e.g., ReLU or Tanh). The output layer 550 provides a classification output such as, for example, identification of the tissue of interest as the detected tissue data 410 (FIG. 4). The activation function used at the output layer 550 may be, for example, the sigmoid function or softmax. CNN 500 may be tuned (learn) to optimize the hyperparameters and/or in any other suitable manner to improve performance thereof. Learning using CNN 500 may be completed prior to deployment for use, e.g., in procedures, or may be updated throughout use in procedures periodically or continuously, using data specific to the system employing CNN 500 or data across multiple systems employing CNN 500. Other suitable machine learning algorithms are also contemplated.

Referring again to FIGS. 1-4, as an alternative to, or in addition to identifying the tissue of interest in the image data, machine learning algorithm 408 may be configured to confirm whether the detection of tissue by controller 140 of tissue detection system 10 was sufficiently accurate. If the detection of tissue by controller 140 of tissue detection system 10 was determined to be accurate, computing system 300 may communicate a confirmatory message to controller 140 of tissue detection system 10 such as, for example, a message indicating that, despite the low confidence in the tissue detection, the tissue detection was determined to be accurate. On the other hand, if the detection of tissue by controller 140 of tissue detection system 10 was determined not to be accurate, computing system 300 may implement machine learning algorithm 408 (or another machine learning algorithm) to detect the tissue (e.g., as detailed above) and return the result to controller 140 of tissue detection system 10 and/or may communicate a reject message to controller 140 of tissue detection system 10 such as, for example, a message indicating that the tissue detection was determined not to be accurate and thus, should not be relied upon.

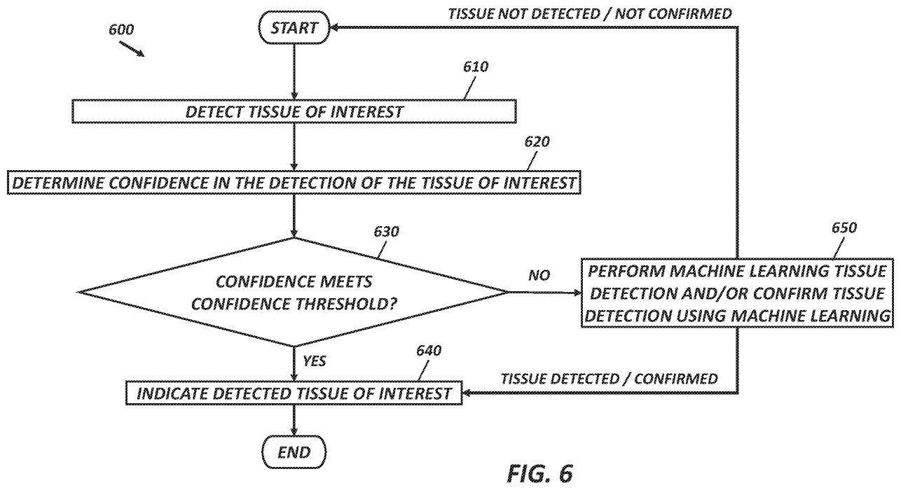

Turning to FIG. 6, a method 600 of detecting tissue in accordance with the present disclosure is illustrated. Initially, at 610, tissue of interest is detected, e.g., using fluorescence as detailed above or in any other suitable manner. At 620, a confidence in the detection of tissue performed at 610 is determined. If the confidence meets or exceeds a confidence threshold, “YES” at 630, the method proceeds to 640 where the detected tissue of interest is indicated to the operator, e.g., via display, highlighting, audible tone, and/or other suitable indication.

If, on the other hand, the confidence does not meet the confidence threshold, “NO” at 630, the method proceeds to 650, wherein machine learning tissue detection is performed and/or the tissue detection is confirmed using machine learning. If the tissue is detected using machine learning tissue detection and/or if the tissue detection is confirmed using machine learning, “TISSUE DETECTED/CONFIRMED” at 650, the method proceeds to 640 where the detected tissue of interest is indicated to the operator, e.g., via display, highlighting, audible tone, and/or other suitable indication. If, on other other hand, the tissue is not detected using machine learning tissue detection and/or if the tissue detection is not confirmed using machine learning, “TISSUE NOT DETECTED/NOT CONFIRMED” at 650, the method reverts to the start, requiring re-starting of method 600 in order to detect the tissue of interest. An error may additionally or alternately be output in this situation.

While several aspects of the disclosure have been shown in the drawings and/or described herein, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope of the claims appended hereto.

What is claimed is:

1. A tissue detection system, comprising:
an emitter configured to generate an electromagnetic radiation signal for application to tissue;
a detector configured to detect fluorescence of the tissue responsive to application of the electromagnetic radiation signal to the tissue; and
a controller configured to detect a tissue of interest within the tissue based upon the detected fluorescence and to determine a confidence associated with the detection of the tissue of interest,
wherein if the determined confidence meets a threshold, the controller is configured to output an indication of the detected tissue of interest, and
wherein, if the determined confidence fails to meet the threshold, a computing device is configured to at least one of: further detect the tissue of interest or confirm the detection of the tissue of interest.

2. The tissue detection system according to claim 1, wherein the computing device is remote from the controller and configured to communicate with the controller over a network.

3. The tissue detection system according to claim 1, wherein the computing device is configured to perform the at least one of the further detecting of the tissue of interest or the confirming of the detection of the tissue of interest via image processing.

4. The tissue detection system according to claim 3, further comprising at least one camera configured to capture an image of the detected tissue of interest, wherein the computing device is configured to perform the at least one of the further detecting of the tissue of interest or the confirming of the detection of the tissue of interest via image processing of the image captured by the at least one camera.

5. The tissue detection system according to claim 4, wherein the at least one camera includes at least one of a visible camera or an infrared (IR) camera.

6. The tissue detection system according to claim 1, wherein the computing device is configured to perform the at least one of the further detecting of the tissue of interest or the confirming of the detection of the tissue of interest using machine learning.

7. The tissue detection system according to claim 1, wherein the computing device is configured to perform the at least one of the further detecting of the tissue of interest or the confirming of the detection of the tissue of interest via machine learning image processing.

8. The tissue detection system according to claim 1, wherein the computing device is a cloud server.

9. The tissue detection system according to claim 1, wherein the confidence is numerical.

10. The tissue detection system according to claim 1, wherein the confidence is categorical.

11. The tissue detection system according to claim 1, further comprising a probe configured to deliver the electromagnetic radiation signal from the emitter to the tissue.

12. The tissue detection system according to claim 11, wherein the probe is coupled between the tissue and the detector to enable the detector to detect the fluorescence of the tissue responsive to the application of the electromagnetic radiation signal to the tissue through the probe.

13. The tissue detection system according to claim 11, wherein the detector is independent of the probe.

14. The tissue detection system according to claim 11, wherein the probe includes a camera, the camera configured to produce image data for use by the computing device in the at least one of the further detecting of the tissue of interest or the confirmation of the detection of the tissue of interest.

15. The tissue detection system according to claim 11, further comprising a camera independent of the probe, the camera configured to produce image data for use by the computing device in the at least one of the further detecting of the tissue of interest or the confirmation of the detection of the tissue of interest.

16. A method of tissue detection, comprising:
applying a signal to tissue;
detecting a response of the tissue from the application of the signal to the tissue;
detecting a tissue of interest within the tissue based upon the detected response;
determining a confidence associated with the detection of the tissue of interest;
if the determined confidence meets a threshold, outputting an indication of the detected tissue of interest; and
if the determined confidence fails to meet the threshold, further detecting the tissue of interest or confirming the detection of the tissue of interest.

17. The method according to claim 16, wherein the detecting is performed locally and wherein the further detecting or the confirming is performed remotely.

18. The method according to claim 16, wherein the detecting is performed with a traditional algorithm and wherein the further detecting or the confirming is performed with a machine learning algorithm.

19. The method according to claim 16, wherein the detecting is performed based on evaluating the response for fluorescence and wherein the further detecting or the confirming is performed based on image processing of an image of the tissue.

20. The method according to claim 19, wherein the signal is an electromagnetic radiation signal, the response is the fluorescence of the tissue, and wherein, if the determined confidence fails to meet the threshold, the method further includes capturing the image of the tissue for the image processing of the image of the tissue.
